(19) United States
(12) Patent Application Publication
(10) Pub. No.: US 2008/0077103 A1
(43) Pub. Date: Mar. 27, 2008

(54) COMPRESSION PACKED ABSORBENT ARTICLE

(76) Inventors: Michelle Klippen, Shakopee, MN (US); Penny Klitzke, Darwin, MN (US)

Correspondence Address: TILLMAN WRIGHT, PLLC, PO BOX 471581, CHARLOTTE, NC, 28247, US

(21) Appl. No.: 11/926,462

(22) Filed: Oct. 29, 2007

Related U.S. Application Data
(63) Continuation-in-part of application No. 10/622,274, filed on Jul. 17, 2003.

(51) Int. Cl. A61F 13/15

(52) U.S. Cl. 604/385.02; 53/434

(57) ABSTRACT

The present invention relates to a package containing a compressed, single, unused absorbent article. The package preserves compression of the absorbent article when sealed. The invention also relates to the method of compressing and packaging a single, unused absorbent article that includes inserting the article into the package, compressing the article, and sealing the package to preserve the compressed state of the article. The compression may be achieved through the use of a vacuum to remove air from the package and absorbent article, or other means of compressing the absorbent article before or after the article is inserted in the package. The absorbent article may be packaged together with other products in a kit. An example kit may be a baby diaper changing kit that includes a single diaper, a changing pad, and wet wipes, and the package maintains at least the compressed state of the diaper.

### CROSS-REFERENCE TO RELATED APPLICATION

The present application is a continuation-in-part of, and claims priority to, U.S. patent application Ser. No. 10/622,274, filed Jul. 17, 2003, which '274 application published as U.S. patent application publication no. US 2005/0015052 A1.

Additionally, the disclosures of U.S. patent application Ser. No. 10/622,274; U.S. patent application publication no. US 2005/0015052 A1; and U.S. patent application publication no. US 2004/0167489 A1 are hereby incorporated herein by reference.

### TECHNICAL FIELD

The present invention generally relates to absorbent articles, and more specifically relates to packaging of a single, unused absorbent article.

### BACKGROUND

Absorbent articles such as diapers are commonly bulky and inconvenient to carry. As a result, many people must carry extra bags or inconveniently large bags simply to accommodate large absorbent articles. Because these articles are often needed away from the home in sometimes unpredictable situations, they must be transportable and easy to use. Furthermore, absorbent articles should be kept clean while in transport to reduce the chances of transmitting disease or illness, which is a major consideration when dealing with many types of absorbent articles.

Various techniques to package bulky sanitary products such as diapers have been developed. However, none of these techniques provide packaging for bulky, individual absorbent articles that are easy for individual consumers to carry and use one at a time.

For example, one packaging technique that has been developed is vacuum packing a plurality of diapers, which compresses the diapers for ease of shipping bulk product. However, the benefit of the vacuum packing is lost when the package is open and a few diapers are removed to carry in a purse or other bag. Another packaging technique has been vacuum packing a soiled diaper after it is used. Packing soiled diapers provides little or no benefit to the individual consumer who needs to pack and carry bulky items such as diapers or other sanitary, absorbent articles. This shortcoming is especially true because most people immediately dispose of soiled diapers and do not carry them in their purses or diaper bags.

Yet other packing techniques have been developed for folding sanitary napkins and packing diapers with wipes and other products. These packaging techniques have limited, if any benefit. Sanitary products tend to be bulky. Folding a product merely changes its dimensions and does not significantly reduce its volume. Additionally, merely placing a wipe in a package with a diaper is convenient, but again, it does not reduce the volume required by the diaper when carrying it in a bag.

### SUMMARY

In general terms, the present invention relates to a sanitary, unused article that is compressed and individually packaged. An individual unused sanitary absorbent article, such as a diaper, may be placed in a compressed state with a small form factor relative to the form factor of the absorbent article in an uncompressed state for convenient handling and transportability of the absorbent article.

Accordingly, one aspect of the claimed invention is a compressed, individually packaged unused absorbent article comprising a sealed package having an internal volume. A single, unused, absorbent article is located in the internal volume. The absorbent article has a compressed state and uncompressed state, and the sealed package holds the absorbent article in the compressed state.

Another aspect of the invention comprises a single, unused, sanitary absorbent article having first and second form factors. The second form factor is substantially smaller than the first form factor. A sealable package has an internal volume sized to receive the absorbent article when in the first form factor. The sealable package is configured to be sealed with the absorbent article maintained in the second form factor inside the package when sealed.

Another aspect of the claimed invention comprises a sealed package formed with an airtight material. A folded diaper has compressed and uncompressed states, and the folded diaper when in the compressed state occupies about 30% to about 70% less volume than when in the uncompressed state. The package maintains the absorbent article in the compressed state. A zip strip is formed in the package allowing easy opening of the package and access to the absorbent article.

Yet another aspect of the claimed invention is a method of compressing and packaging a single unused sanitary absorbent article in a sealable, compressible package. The method comprises inserting a single unused sanitary absorbent article into the package; compressing the absorbent article to reduce the size of the absorbent article; and sealing the package, the sealed package maintaining the article in a compressed state.

### DESCRIPTION OF THE DRAWINGS

FIG. 1 is a perspective view of a compression packaged absorbent article according to principles of the invention.

FIG. 2 is a side view of the compression packaged absorbent article shown in FIG. 1.

FIG. 3 is a side view of one example folding arrangement for an absorbent article using two folding axes.

FIG. 4 is a side view of another example folding arrangement for an absorbent article using two folding axes.

FIG. 5 is a side view of a rolled absorbent article.

FIG. 6 is a side view of yet another example folding arrangement for an absorbent article using three folding axes.

FIG. 7 is an exploded perspective view of the components of the vacuum packaged absorbent article shown in FIG. 1.

FIG. 8 is a cross-sectional side view of the compression packaged absorbent article shown in FIG. 1 before the absorbent article has been compressed.

FIG. 9 is a perspective view of an example compression packaged diaper kit that includes a diaper, a wet wipe, and a changing pad according to principles of the invention.

### DETAILED DESCRIPTION

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention. There are alternative embodiments for all of the structures and methods disclosed herein regardless of whether specific alternatives are set forth.

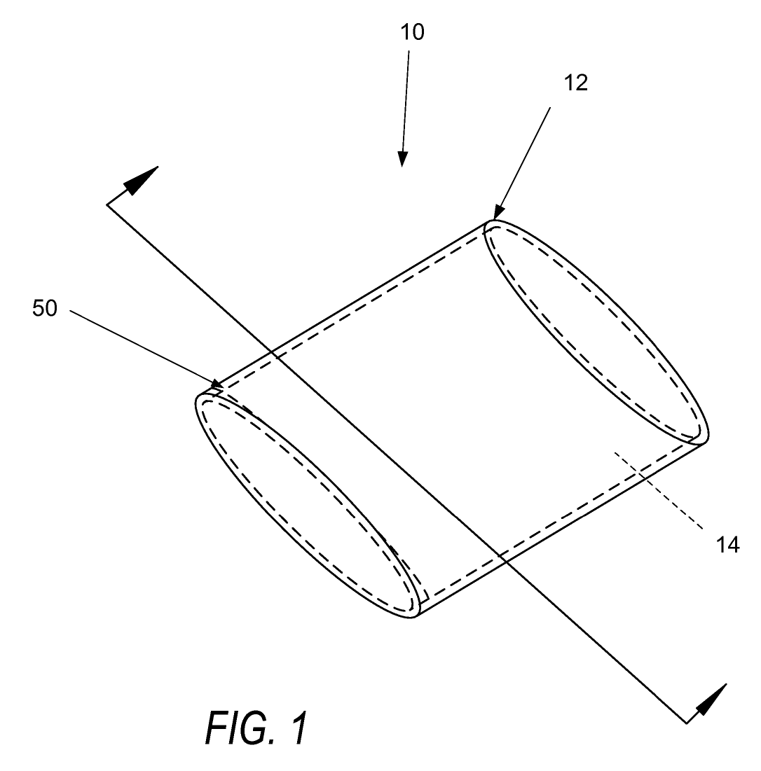

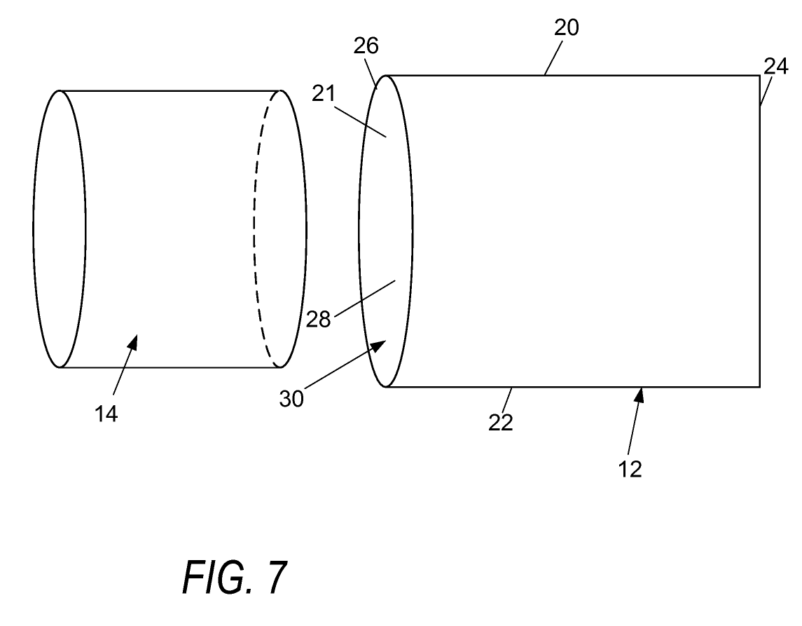

Referring to FIGS. 1 and 7, a packaging material 12 includes first and second opposing side portions 20 and 22 and has first and second ends 24 and 26. The packaging material defines an internal volume 30. A single, unused absorbent article is positioned within the internal volume 30 of the packaging material 12. In one possible embodiment, the packaging material 12 is sealed so that internal volume 30 is entirely enclosed or completely surrounded and isolated from the area external to the packaging materials.

The packaging material 12 can be formed with a variety of different configurations. In at least some embodiments, for example, the first and second side portions 20 and 22 are formed from separate flat panels. In yet other possible embodiments, the first and second side portions 20 and 22 are formed from a single, tubular sheet of material having an endless wall.

The packaging material 12 can be formed with a variety of different materials. In one possible embodiment, the packaging material 12 is formed with a material that is substantially impermeable to water. An advantage of this embodiment is that it protects the absorbent article if the package is inadvertently exposed to moisture. In another possible embodiment, the material used to form the packaging material 12 is substantially impermeable to air. This embodiment enables a vacuum to be drawn and maintained in the interior volume thereby allowing the first and second side portions 20 and 22 of the packaging material 12 to collapse and compress the single, unused absorbent article 14.

Additionally, the packaging material 12 includes a selectively openable region 50 that allows a user to easily open the packaging material 12 and access the single, unused absorbent article 14. The selectively openable region 50 can have a variety of different structures. Examples include a score, perforation line, pull strip, resealable zip strip, or other structure that facilitates easy opening of the packaging material 12 for access to the internal volume 30 of the packaging 12. In at least some possible embodiments, the openable region 50 does not compromise the sealed or resealable nature of the packaging material 12, which allows a vacuum to be drawn in the interior volume and also prevents moisture from entering the interior volume 30. Additionally, in at least some possible embodiments, the openable region 50 is resealable, which may be advantageous, for example, to repackage the absorbent article 14 after use of the article.

The openable region 50 may be positioned on either of the side portions 20 and 22 of the package material or on either end 24 or 26 of the packaging material 12. Additionally, the openable region 50 can have any orientation. It may run the width or the length of the package. Additionally, the openable region may have any length. It may extend for the entire length or the packaging material, the entire width of the packaging material, or any portion thereof. Additionally, the openable region may extend entirely around the packaging material and form an endless line.

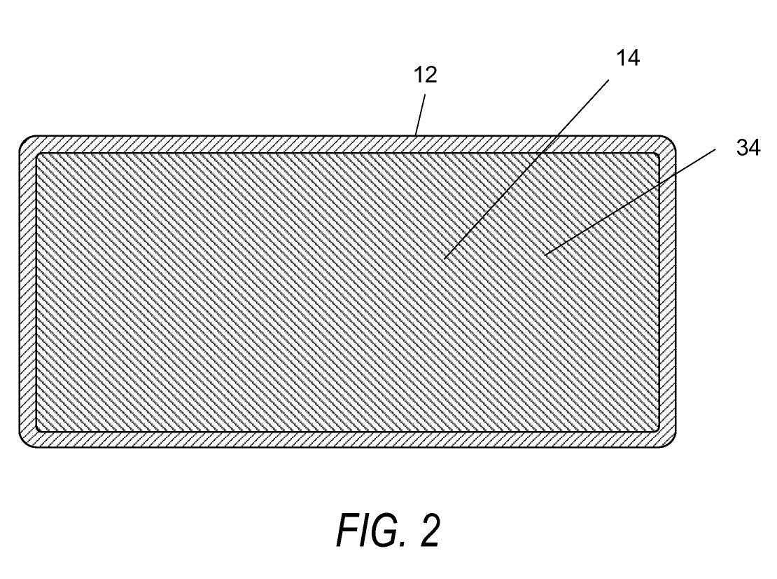

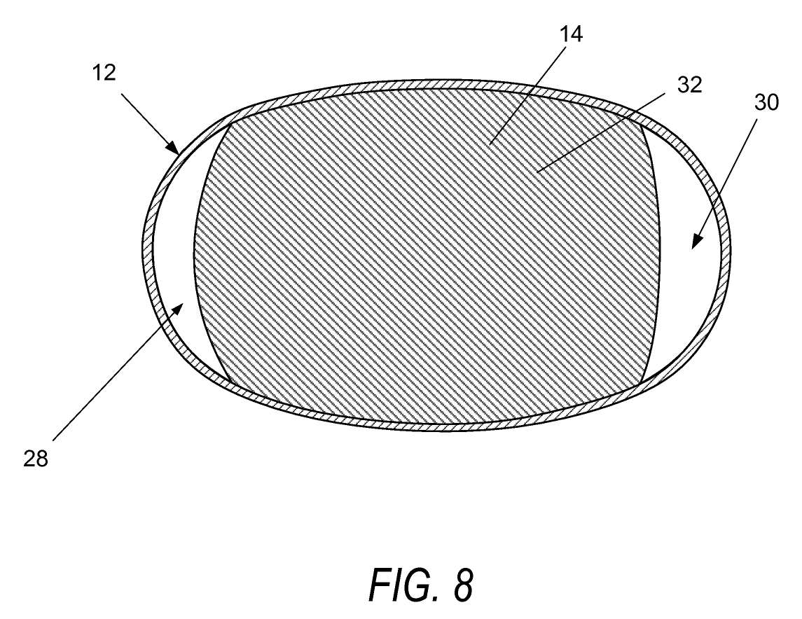

The single, unused absorbent article 14 has first and second states 32 and 34. The first state 32 (FIG. 8) is an uncompressed state in which the single, unused absorbent article has its natural form factor and can be used for its intended purpose. The second state 34 (FIG. 2) is a compressed state in which the form factor or overall volume of the single, unused absorbent article 14 is reduced so that it can be carried and stored while taking up minimal storage space. The packaging material 12 holds the single, unused absorbent article 14 in the second state until a user selectively removes the single, unused absorbent article 14 from the internal volume 30.

Examples of the articles that can form the single, unused absorbent article 14 include children's and adult diapers, sanitary napkins and other feminine hygiene products, first aid and other medical products, or any other absorbent article that a consumer might want to carry. The term diaper is used herein for ease of illustration and description.

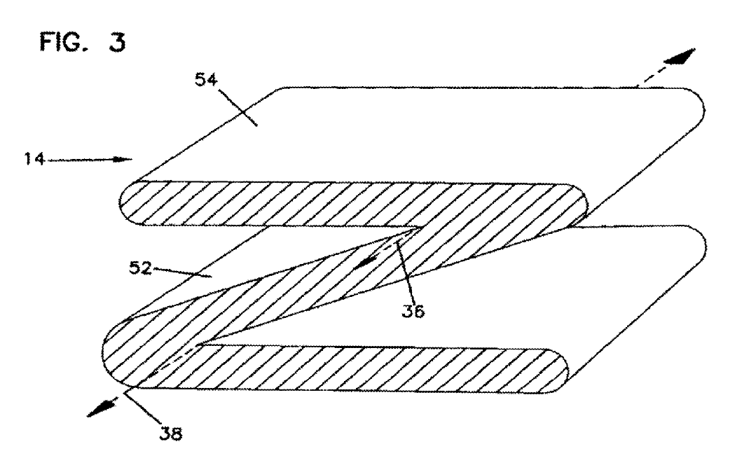

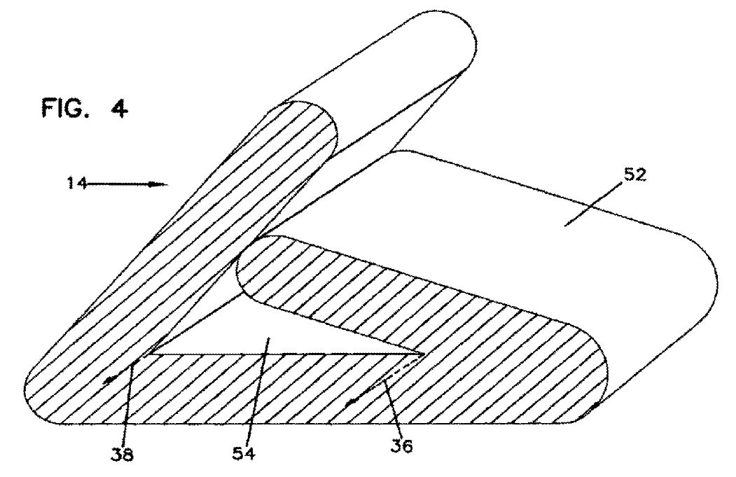

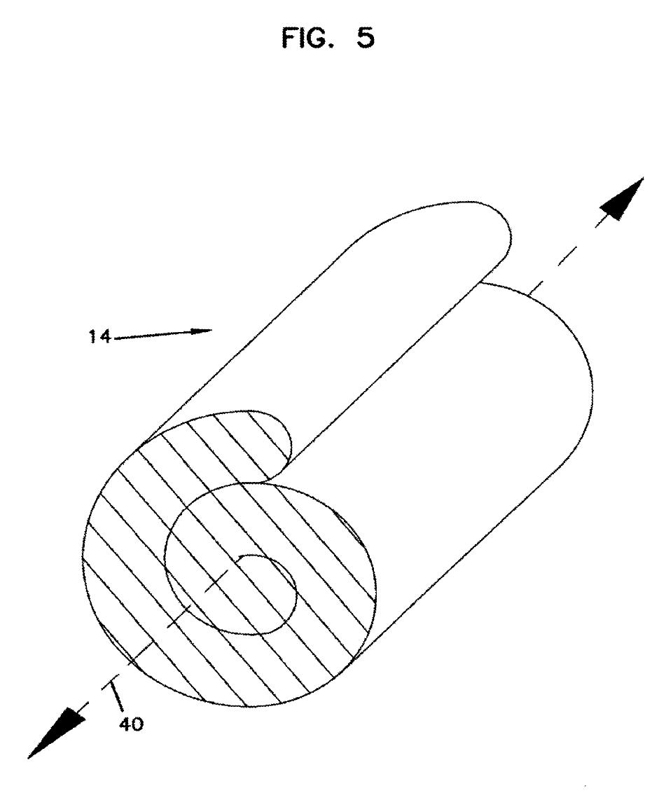

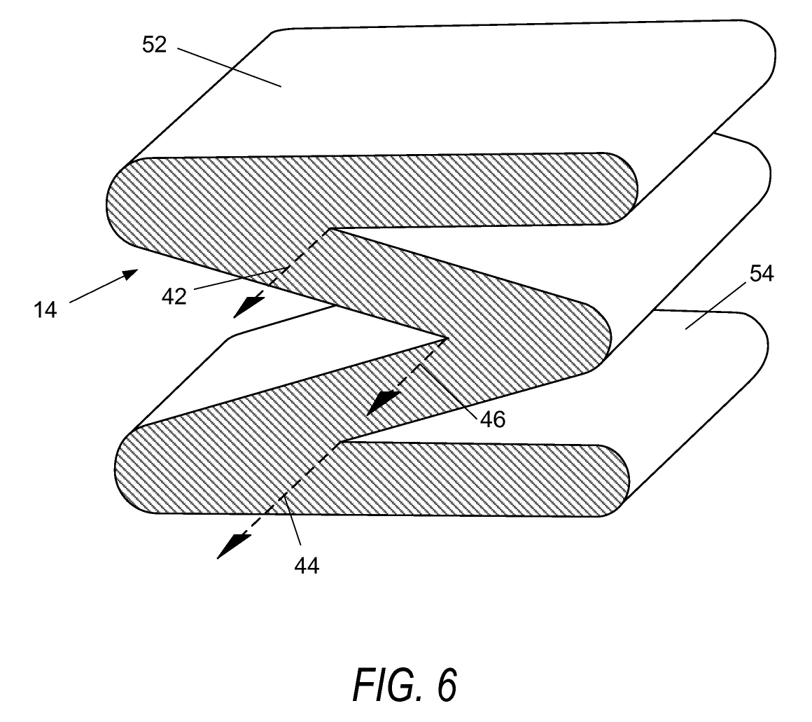

Additionally, the single unused diaper 14 is folded when positioned in the interior volume. Referring to FIG. 3, for example, one possible folding arrangement folds the single, unused diaper 14 about two fold axes 36, 38. The diaper includes first and second primary surfaces 52, 54 that are folded over each other along the fold axes 36, 38 to form a generally “Z” shaped folding arrangement. Referring to FIG. 4, another possible folding arrangement involves folding the single, unused diaper about two fold axes 36, 38. In this configuration, the primary surfaces 52, 54 are folded about fold axes 36, 38 to form a generally “C” shaped folding arrangement. FIG. 5 illustrates yet another folding arrangement for the single, unused diaper 14 in which the diaper 14 is rolled about a single axis 40. FIG. 6 illustrates a folding arrangement in which the single, unused diaper is folded about three fold axes 42, 44, and 46. In this arrangement, the primary surfaces 52, 54 are folded over each other twice to form a generally “M” shaped folding arrangement.

Referring to FIGS. 7 and 8, the single, unused absorbent diaper 14 and the packaging material 12 can be assembled in a variety of different ways. In at least some possible embodiment, the single, unused absorbent diaper 14 and the packaging material 12 are assembled using a vacuum. In these embodiments, the packaging material 12 forms an opening 28, and the internal volume 30 is sized to receive the single, unused absorbent diaper 14 when it is folded, but still in the first, uncompressed state 32. In this embodiment, the interior volume is slightly larger than the single, unused absorbent diaper 14 when it is in a state (e.g., folded, semi-compressed, or fully compressed) ready to be inserted into the packaging material. The single, unused absorbent diaper 14 is folded and then inserted into the interior volume 30. A vacuum is then drawn in the interior volume 30, which causes the side portions 20 and 22 to collapse toward one another and compress the single, unused sanitary diaper 14 into a smaller form factor so that it occupies a smaller volume. The opening 28 is then sealed to maintain the vacuum in the interior volume 30.

There are many other possible ways to assemble the single, unused absorbent diaper 14 and the packaging material 12. The single, unused absorbent diaper 14 can be compressed into the second state 34 and then is inserted into the interior volume 30. The single, unused absorbent diaper 14 is allowed to expand to the dimensions of the interior volume 30 as defined by the first and second opposing side portions 20 and 22. In this embodiment, a vacuum is not used to place the single, unused absorbent diaper 14 into the second, compressed state 34. Additionally, in this embodiment, the packaging material 12 may not be sealed. For example, the first and second ends 24 and 26 can be open so that the packaging material 12 is generally tubular in configuration.

In another possible embodiment, the packaging material 12 is formed with a heat shrink material. In this embodiment, the single, unused absorbent diaper 14 is inserted into the interior volume 30 and then the packaging material 12 is heated causing it to shrink and compress the single, unused absorbent diaper 14 into the second state 34. Again, in this embodiment, the packaging material 12 may not be sealed.

Additionally, in other embodiments, these basic assembly techniques can be combined. For example, assembly may include the actions of compressing the single, unused absorbent diaper 14 into a compressed or semi-compressed state, inserting the compressed or semi-compressed single, unused absorbent diaper 14 into the interior volume 30, and then drawing a vacuum. Many other embodiments and combinations of actions are possible while assembling the single, unused absorbent diaper 14 and the packaging material 12.

As demonstrated in the following examples, the packaging of a single, unused diaper 14 as described results in a substantial reduction in the diaper's form factor or volume. In one possible embodiment, for example, the volume from the first, uncompressed state 32 to the second, compressed state 34 is reduced by about 40% or more. The following examples set forth the experimental results of packaging various sizes and brands of diapers with a compressive vacuum pressure of 0.035 megapascals (MPa). Although certain brands and sizes were tested at this pressure, it is understood that the packaging techniques and structure disclosed herein can be used with any size and type of single absorbent article with any type and amount of compressive force sufficient to compress the single absorbent article and reduce its volume.

### EXAMPLE 1

A size 2 children's Pampers™ diaper has a length of 7.3 inches, a width of 4.3 inches, and a height of 0.7 inches in an uncompressed state (first form factor), and a length of 4.4 inches, width of 3.0 inches, and height of 0.8 inches in a compressed state (second form factor). As a result, the first form factor has a volume of about 22.8 inches cubed and the second form factor has a volume of about 10.1 inches cubed, which is about a 55.54% reduction in volume from the first to the second form factor.

### EXAMPLE 2

A size 5 children's Huggies™ diaper has a length of 9.5 inches, a width of 4.3 inches, and a height of 0.7 inches in an uncompressed state (first form factor), and a length of 2.9 inches, width of 4.3 inches, and height of 0.9 inches in a compressed state (second form factor). As a result, the first form factor has a volume of about 29.4 inches cubed and the second form factor has a volume of about 11.1 inches cubed, which is about a 62.29% reduction in volume from the first to the second form factor.

### EXAMPLE 3

An adult extra-large Depends™ diaper has a length of 8.6 inches, width of 5.7 inches and height of 1.6 inches in an uncompressed state (first form factor), and a length of 5.2 inches, width of 4.1 inches, and height of 1.2 inches in a compressed state (second form factor). As a result, the first form factor has a volume of about 77.5 inches cubed and the second form factor has a volume of about 25.5 inches cubed, which is about a 67.15% reduction in volume from the first to the second form factor.

### EXAMPLE 4

An adult medium-sized Depends™ diaper has a length of 9.5 inches, width of 5 inches and height of 0.88 inches in an uncompressed state (first form factor), and a length of 4.24 inches, width of 2.72 inches, and height of 1.5 inches in a compressed state (second form factor). As a result, the first form factor has a volume of about 41.8 inches cubed and the second form factor has a volume of about 17.3 inches cubed, which is about a 58.61% reduction in volume from the first to the second form factor.

Other examples have changes in volume between the first and second form factors of greater than 30%, with changes in volume of about 80% to 90% being possible in some cases of highly porous absorbent materials, such as cotton balls and sponges. The percentage of reduced volume is typically dependent on the article being compressed. Thus, in one possible embodiment, the change in volume between first and second form factors is in the range from about 30% to about 70%. In another embodiment the change in volume is about 30% or more. Another embodiment has a change in volume of about 40% or more. Another embodiment has a change in volume of about 55% or more.

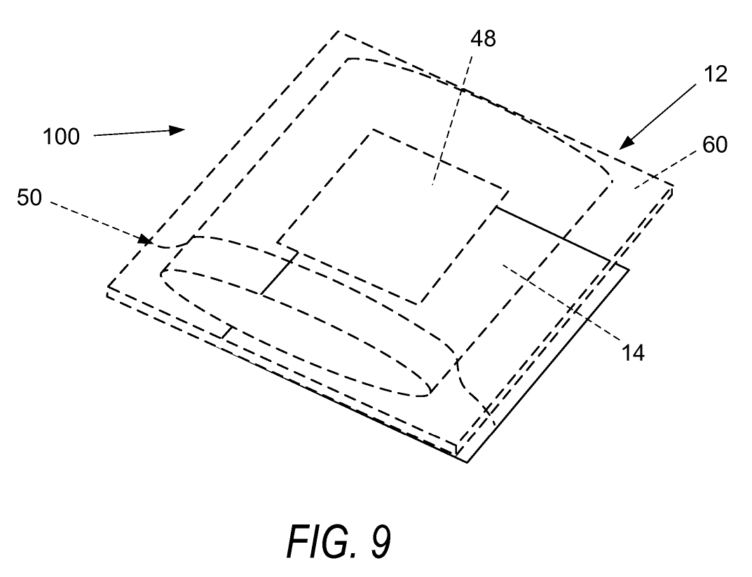

Referring to FIG. 9, at least some possible alternative embodiments may include kits having an item complimentary to the single, unused absorbent article. For example, the kit 100 may include one or more wipes 48 positioned in the interior volume together with a single, unused diaper 14. In alternative embodiments, the wipes 48 may be individually wrapped or may be located between moisture impermeable surfaces of the diaper 14. Other products may also be added to the package, such as, for example, a bag 60 for disposal of the diaper after it is used, packaged lotion or ointments, and deodorizers. In other embodiments, some of the complementary items held in the packaging 12 are compressed, while other items are uncompressed when the packaging material 12 is sealed.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention, which is set forth in the following claims.

What is claimed is:

1. A method of compressing and packaging a single unused disposable diaper, the method comprising:
(a) compressing a single unused diaper to reduce a size of the diaper, whereby the diaper exists in a compressed state;
(b) locating the single unused diaper while in the compressed state within a package, the package comprising a heat shrinkable material;
(c) drawing a vacuum within an interior of the package such that, when exposed to atmospheric pressure, portions of the package will collapse toward and compress the diaper;
(d) heating the package to shrink wrap the heat shrinkable material about the diaper; and
(e) sealing the package to maintain the vacuum within the interior of the package.

2. The method of claim 1, wherein said step (d) is performed prior to said step (e).

3. The method of claim 1, wherein said step (e) is performed prior to said step (d).

4. The method of claim 1, wherein said step (e) is performed during performance of said step (d).

5. The method of claim 1, wherein the package comprises a tubular sheet of material having an endless wall.

6. The method of claim 1, wherein the package comprises separate flat panels of material which are sealed in said step (e) to form the airtight interior space of the package.

7. The method of claim 1, wherein said step (b) comprises drawing a vacuum at a pressure of about 350 millibars.

8. The method of claim 1, wherein a volume of the absorbent article is reduced by the method by about 30% to about 70%.

9. A method of packaging an absorbent article, the method comprising:
(a) disposing an absorbent article within an interior volume defined by a heat-shrinkable material; and
(b) heating the heat-shrinkable material thereby shrinking the heat-shrinkable material about the absorbent article;
(c) wherein the absorbent article is the only absorbent article disposed within the interior volume defined by the heat shrinkable material;
(d) wherein the absorbent article is unsoiled;
(e) wherein the absorbent article, prior to packaging, has an uncompressed state; and
(f) wherein the absorbent article, after said step (b), has a compressed state that has a lesser volume than that of the uncompressed state.

10. The method of claim 9, wherein said step (b) comprises heating the heat-shrinkable material thereby shrinking the heat-shrinkable material about the absorbent article and reducing the volume of the absorbent article to the compressed state.

11. The method of claim 9, further comprising compressing the absorbent article from the uncompressed state prior to said step (a).

12. The method of claim 9, wherein said step (a) comprises disposing the absorbent article in the compressed state within the interior volume defined by the heat-shrinkable material.

13. The method of claim 12, wherein said step (b) comprises heating the heat-shrinkable material thereby shrinking the heat-shrinkable material about the absorbent article and retaining the absorbent article in the compressed state.

14. A compressed, individually packaged unused diaper comprising: a sealed package comprised of airtight material; a folded diaper having compressed and uncompressed states wherein the compressed state occupies about 30% to about 70% less volume than the uncompressed state and the package maintains the absorbent article in the compressed state. a selectively openable region in the package allowing easy opening of the package and access to the absorbent article.

15. A method of compressing and packaging a single unused sanitary absorbent article in a sealable, compressible package, the method comprising: inserting a single unused sanitary absorbent article into the package; compressing the absorbent article to reduce the size of the absorbent article; and sealing the package, the sealed package maintaining the article in a compressed state.

16. A compressed, individually packaged unused absorbent article, comprising: a single, unused, sanitary absorbent article having first and second form factors, the second form factor being substantially smaller than the first form factor; and a sealable package having an internal volume sized to receive the absorbent article when in the first form factor, and configured to be sealed with the absorbent article maintained in second form factor inside the package when sealed.

17. A compressed, individually packaged unused absorbent article, comprising: a sealed package having an internal volume; and a single, unused, absorbent article located in the internal volume, the absorbent article having a compressed state and uncompressed state, the sealed package holding the absorbent article in the compressed state.

18. The packaged absorbent article according to claim 17, wherein the absorbent article is a compressible feminine hygiene product.

19. The packaged absorbent article according to claim 17, wherein such compression reduces the volume of the packaged absorbent article by about 30% to about 70%.

20. The packaged absorbent article according to claim 17, wherein the absorbent article is folded together about two fold axes while in the uncompressed state.

21. The packaged absorbent article according to claim 17, wherein the absorbent article is folded together about three fold axes while in the uncompressed state.

22. The packaged absorbent article according to claim 17, wherein the absorbent article is rolled while in the uncompressed state.
